(19) United States
(12) Patent Application Publication
(10) Pub. No.: US 2009/0030088 A1
(43) Pub. Date: Jan. 29, 2009

(54) Therapeutic benefits of gossypol, 6-methoxy gossypol, and 6,6'-dimothxy gossypol

(75) Inventors: Xi Wang, Central, SC (US); James C. Morris, Clemson, SC (US); Feng Chen, Central, SC (US); Thomas Beckham, Charleston, SC (US)

Correspondence Address: J. BENNETT MULLINAX, LLC, P. O. BOX 26029, GREENVILLE, SC, 29616-1029, US

(73) Assignee: Clemson University, Clemson, SC (US)

(21) Appl. No.: 12/148,574

(22) Filed: Apr. 21, 2008

Related U.S. Application Data
(60) Provisional application No. 60/925,516, filed on Apr. 20, 2007.
(60) Provisional application No. 61/072,565, filed on Apr. 1, 2008.

(51) Int. Cl. A61K 31/11; A61P 31/00; A61P 35/00

(52) U.S. Cl. 514/700

(57) ABSTRACT

6-methoxy gossypol and 6,6′-dimethoxy gossypol were isolated from cottonseeds. Bioactivities of these two gossypol derivatives and gossypol were investigated regarding their antioxidant activities, DNA damage prevention ability, anti-cancer, and anti-trypanosomal activities. Both methoxy compounds had nearly equivalent bioactivities, but gossypol showed greater bioactivities than either methoxy derivative on free radical scavenging activity, reducing power, and DNA damage prevention ability. Gossypol and its methoxy derivatives inhibited growth of three cancer cell lines, i.e., SiHa (cervical cancer), MCF-7 (breast cancer) and Caco-2 (colon cancer) cells, in a dose dependent manner. These three compounds also significantly inhibited growth of trypanosome T. brucei, the cause of African Sleeping Sickness, which affects thousands in western and central Africa.

### RELATED APPLICATIONS

This application claims the benefit of U.S. Application Ser. No. 60/925,516 filed on Apr. 20, 2007, and U.S. Application Ser. No. 61/072,565, filed on Apr. 1, 2008, both of which are incorporated herein by reference.

### FIELD OF THE INVENTION

This invention is directed to the use of gossypol and gossypol derivatives which have been found to significantly inhibit the growth of trypanosome (T. brucei) the causative agent of African Sleeping Sickness. Additionally, the invention relates to bioactivity of gossypol and gossypol derivatives with respect to antioxidant properties and other biological activities.

### BACKGROUND OF THE INVENTION

Cottonseed contains a considerable amount of gossypol, from 1.7% to occasionally 6% of its dry weight. When cottonseed is processed for making cottonseed oil, gossypol and gossypol derivatives may be remained in large quantities as waste in soap stock or in cottonseed meal.

In recent years, gossypol has attracted much attention because of its potential anti-proliferative activities on a variety of human cancer cells, including hormone-dependent human breast, colon, and prostate cancers (1-4). Some studies have demonstrated that gossypol-induced cell growth inhibition involves in both cell cycle arrest and apoptosis by activating transforming growth factor-b(15,16) upregulating P53 and P21, and down-regulating cyclin D1 and Rb. However, some studies pointed out that the in vitro antitumor activity of gossypol was weakened by the presence of serum, possibly due to the formation of Schiff base between gossypol and protein in the serum (5, 6). This suggested that the functional groups of gossypol might play important roles on some bioactivities. Accordingly, there remains room for improvement and variation within the art of gossypol and derivatives of gossypol.

### SUMMARY OF THE INVENTION

It is an aspect of at least one embodiment of the present invention to provide for hydroxyl modified gossypol derivatives for use as an anti-trypanosomal treatment protocol.

It is a further aspect of at least one embodiment of the present invention to provide for methoxy derivatives of gossypol that may be used as antioxidant additives for food.

It is yet a further aspect of at least one embodiment of the present invention to provide for methoxy gossypols having higher cancer inhibitive activity than modified gossypol in cervical, breast, and colon cancer cell lines.

It is yet a further aspect of at least one embodiment of the present invention to provide for a process of treating a trypanosome infection comprising the steps of providing a patient infected with trypanosome; introducing into the patient an effective amount of at least one of a gossypol, 6-methoxy gossypol, 6,6′-dimethoxy gossypol or combinations thereof, thereby removing trypanosomes from the patient.

It is yet a further aspect of at least one embodiment of the present invention to provide for a method for a treatment of a cancer selected from cervical cancer, breast cancer, and colon cancer in a mammal, which comprises administering to a mammal in need of such treatment an effective amount of a compound as seen in FIG. 1 wherein R₁ and R₂ are each independently selected from the group consisting of H, and CH₃.

These and other features, aspects, and advantages of the present invention will become better understood with reference to the following description and appended claims.

### BRIEF DESCRIPTION OF THE DRAWINGS

A fully enabling disclosure of the present invention, including the best mode thereof to one of ordinary skill in the art, is set forth more particularly in the remainder of the specification, including reference to the accompanying drawings.

FIG. 1 sets forth the structure and functional groups of gossypol, 6,6′-dimethyoxy gossypol, and 6-methoxy gossypol.

FIG. 2 is a graph showing free radical scavenging activity of gossypol and methoxy derivatives of gossypol.

FIG. 3 is a graph showing the reducing power of gossypol, methoxy derivatives of gossypol, and the food grade antioxidant BHT.

FIGS. 4A and 4B provide an analysis of DNA strand breakage as demonstrated by gel electrophoresis of a strand scission assay.

FIGS. 5A through 5C set forth cancer cell viability assays for gossypol and methoxy derivatives of gossypol.

FIG. 6 is a graph showing relative cell viability of trypanosome cultures for gossypol and methoxy gossypol derivatives.

### DETAILED DESCRIPTION

Reference will now be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used on another embodiment to yield a still further embodiment. Thus, it is intended that the present invention cover such modifications and variations as come within the scope of the appended claims and their equivalents. Other objects, features, and aspects of the present invention are disclosed in the following detailed description. It is to be understood by one of ordinary skill in the art that the present discussion is a description of exemplary embodiments only and is not intended as limiting the broader aspects of the present invention, which broader aspects are embodied in the exemplary constructions.

In describing the various figures herein, the same reference numbers are used throughout to describe the same material, apparatus, or process pathway. To avoid redundancy, detailed descriptions of much of the apparatus once described in relation to a figure is not repeated in the descriptions of subsequent figures, although such apparatus or process is labeled with the same reference numbers.

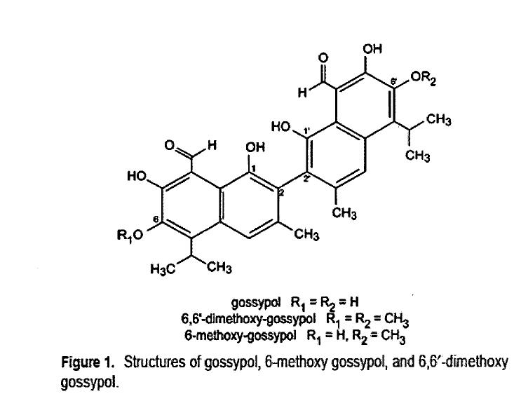

6-Methoxy gossypol, and 6,6′-methoxy gossypol, for which the hydroxyl group(s) of gossypol are methyl esterified, have been found in certain cotton species (7). It is known that the loss of certain hydroxyl groups in gossypol may cause significant changes on bioactivities, however, relevant studies of methoxy gossypol derivatives are rather meager due to their limited availability. Based on the successful isolation of 6-methoxy gossypol and 6,6′-dimethoxy gossypol from Gossypium barbadense Sea Island cotton by using a preparative chromatographic technique (8), a sufficient amount of methoxy gossypol derivatives were provided for this study, which aimed to investigate and compare the bioactivities of gossypol, 6-methoxy-gossypol and 6,6′-dimethoxy-gossypol (FIG. 1) regarding their free radical scavenging activity, reducing power, DNA damage prevention ability, anti-tumor and anti-trypanosomal activities.

As seen in reference to FIG. 1, a core structure of gossypol in which R₁ and R₂ are hydrogen. As seen in reference to FIG. 1, 6,6′-dimethoyx gossypol module is provided where R₁═R₂ which equals CH₃. Further, the 6-methoxy gossypol is provided by the structure where R₁═H and R₂═CH₃.

In addition to the above-identified derivatives of gossypol, it is believed that the R group can be comprised of a number of different functional molecules including various polysaccharide, disaccharide, monosaccharide, chloride, phosphate, fluoride, amines, sulfate, and other polyfunctional groups. The assays described herein can be readily used as screening protocols to determine the efficacy and effectiveness of various other derivatives where one or more of the R groups may have the substitutions described above and combinations thereof.

Materials. Sodium pyruvate, sterile cell culture penicillin-streptomycin, Rosewell Park Memorial Institute 1640 (RPMI 1640), sodium biocarbonate, non-essential amino acid, and trypsin-EDTA solution, gossypol (acetic acid), 2,2′-diphenyl-1-picrylhydrazyl (DPPH) radical, and butylated hydroxytoluene (BHT) were purchased from Sigma Chemical Co. (St. Louis, Mo.). Tissue culture plates were from Costar (Cambridge, Mass.). Heat inactivated fetal bovine serum, fetal bovine serum and newborn calf serum were purchased from Hyclone (Logan, Utah). Dichloromethane (DCM), acetone and trichloroacetic acid were purchased from Fisher Scientific (Suwanee, Ga.). Potassium ferricyanide was obtained from J. T. Baker Chemical Co. (Phillipsburg. N.J.). 6-Methoxy gossyspol and 6,6-dimethoxy gossypol were prepared as described by Dowd and Pelitire (8).

Determination of Antioxidant Activity. The antioxidant capacities of gossypol, 6-methoxy gossypol and 6,6′-dimethoxy gossypol were assessed by two methods: the DPPH free radical scavenging assay and a reducing power assay.

DPPH Free Radical Scavenging Assay. Scavenging activities on DPPH free radicals by gossypol and methoxy gossypol derivatives were determined according to the method of (9) with slight modification. The reaction mixture was made by mixing 0.4 mL of sample solution in DCM and same volume of 0.25 mM DPPH in DCM, shaken vigorously, incubated for 30 min in the dark at room temperature, and measured spectrophotometrically at 517 nm. BHT, a common used antioxidant, was used as a standard for comparison. The lower absorbance of reaction mixture indicated higher free radical scavenging activity, and the reduction of DPPH free radicals was calculated as following:

$$Scavenging activity ( \% ) = ( 1 - \frac{absorbance of sample at 517 nm}{absorbance of control at 517 nm} ) \times 100 \qquad ( 1 )$$

Reducing Power Assay. The reducing power of gossypol and methoxy gossypol derivatives was determined according to the method of Chung et al (10). An aliquot of 0.5 mL of gossypol or methoxy gossypol acetone solution was mixed with 1 mL of 1% potassium ferricyanide [K₃Fe(CN)₆], and incubated at 50° C. for 20 min. Then 1 mL of trichloroaceteic acid (10%) was added, and the mixture was centrifuged at 3000 rpm for 10 min. The upper layer of the solution (1 mL) was mixed with distilled water (1 mL) and FeCl₃ (0.2 mL, 0.1%), and measured spectrophotometrically at 700 nm. Higher absorbance of the reaction mixture indicated higher reducing power.

Analysis of DNA Strand Breakage. The DNA strand scission assay was performed as described by Keum et al., (11) with minor modifications. The plasmid DNA was prepared and purified from E. coli cultures by the method (12). The reaction mixture (15 μL) contained 10 mM Tris-HCl, 1 mM EDTA buffer (pH 8.0), plasmid DNA (1 μL), and H₂O₂ (0.04 M). Gossypol and methoxy gossypol derivatives dissolved in DMSO at the defined concentrations were added prior to H₂O₂ addition. Hydroxyl radicals were generated by irradiation of the reaction mixture at a distance of 30 cm with a 12 W UV lamp. After incubation at room temperature for 30 min, the reaction was stopped by adding a loading buffer (0.25% bromophenol blue tracking dye and 40% sucrose), and analyzed by 1% agarose gel electrophoresis. The gel was visualized by staining with ethidium bromide, and photographed on a transiluminator (Biorad).

Anticancer Activities. Three cancer cell lines, MCF-7 (human breast cancer cell line), Caco-2 (human colon cancer cell line) and SiHa (cervical cancer cell line) were purchased from American Type Culture Collection (ATCC) (Rockville, Md.). MCF-7 and SiHa were cultured in RPMI-1640 with L-glutamine (2 mM), sodium pyruvate (1 mM), penicillin (100 unit/mL), streptomycin (0.1 mg/mL), 0.1 mM non-essential amino acids, 2.0 g/L sodium bicarbonate and 10% newborn calf serum. For Caco-2 cells, 10% fetal bovine serum was added instead of 10% newborn calf serum. All cell lines were incubated at 37° C. with 5% CO₂ and 90-100% relative humidity. Medium renewal was carried out 2-3 times per week, and cells were subcultured when they were about 80-90% confluence.

Prior to chemical treatment, 10⁴ cells/well (100 μL) were seeded into a 96-well tissue culture plate, and allowed to attach for 24 hours, then treated with defined concentrations of the tested chemicals in DMSO. Negative controls were cells treated with DMSO only, and DMSO concentration was kept 2% in each well. After 24 hour incubation, cell proliferation was determined using the CellTiter 96® aqueous nonradioactivity cell proliferation assay according to the manufacture's recommendations (Promega, Madison, Wis.) and recorded on universal EL800 Bio-Tek microplate reader at 490 nm.

Anti-trypanosomal activities. Trypanosome brucei cells were grown in HMI-9 medium supplemented with 10% heat-inactivated fetal bovine serum and cultured as described by (13). Prior to treatment, 200 μL of the cells were seeded into 96-well tissue culture plate and treated with 2 μL of gossypol or methoxy gossypol derivatives dissolved in DMSO. After 24 hour incubation at 37° C. with 5% CO₂ and 90-100% relative humidity, the cells were counted on a Becton Dickinson FACScan flow Cytometer, and the relative cell viability was calculated by comparing the vital cell number with control wells treated only with DMSO. Statistical Analysis. Each experiment was done at least three times, mean values were average of the triplicates, and the data were subjected to the analysis of variance (ANOVA).

### RESULTS AND DISCUSSION

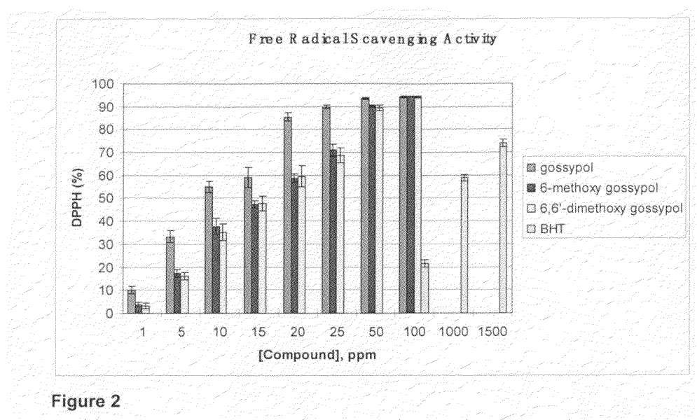

Free Radical Scavenging Activity. It has been reported that the free radical scavenging activities by radical scavengers may vary upon the use of protic or aprotic solvents (14). In this study, DCM was chosen as the solvent of gossypol and methoxy gossypol derivatives for the DPPH free radical scavenging activity test with BHT as a reference. The concentrations of gossypol, 6-methoxy gossypol and 6,6′-dimethoxy gossypol to scavenge 50% free radicals (IC₅₀ value) are 8.2 ppm, 16.4 ppm and 16.8 ppm, respectively (FIG. 2). Though 6-methoxy gossypol exhibited similar free radical scavenging activity with 6,6′-dimethoxy gossypol, gossypol possessed a stronger radical scavenging activity than its derivatives. Such radical scavenging differences between phenolic compounds might depend greatly on the number, arrangement and esterification of phenolic hydroxyl groups. Our study indicated that the methylation of one or two —OH groups on the naphthyl ring of gossypol greatly decreased the ability to quench the free radicals. Nevertheless, compared with the commercial antioxidant BHT, gossypol and the methoxy gossypols showed much greater free radical scavenging activities (FIG. 2). For example, gossypol at 20 ppm could scavenge 85% of free radicals, which is greater than that (75%) of BHT at 1500 ppm. Similarly, 20 ppm of methoxy gossypol or dimethoxy gossypol showed comparable free radical scavenging activity (60%) with 1000 ppm of BHT.

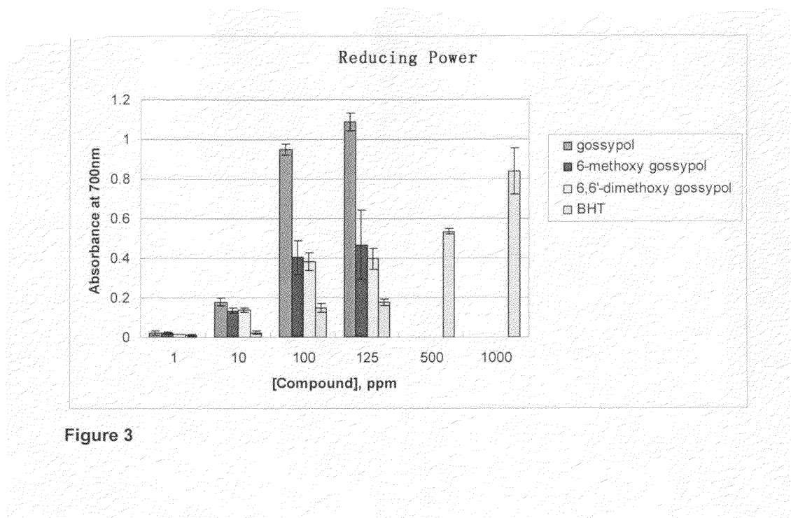

Reducing Power. As shown in FIG. 3, gossypol, 6-methoxy gossypol and 6,6′-methoxy gossypol reduce ferric to ferrous in a dose depended manner within the tested range 1-125 ppm. 125 ppm is the highest concentration that can be tested because of the solubility restraint of gossypol and (di)-methoxy gossypol in the test system. Like the case in the DPPH test, gossypol showed remarkably greater reducing power and higher efficiency than methoxy gossypol and dimethoxy gossypol, though both gossypol and its derivatives showed much greater reducing power than BHT. For instance, gossypol and its derivatives within the concentration ranges of 1 to 125 ppm showed significantly higher reducing power than BHT at the same concentration, and (di)methoxy gossypol at 10 ppm has similar reducing power to 100 ppm of BHT. Regardless of the negative effect of methylation of —OH groups on gossypol that decreased both the free radical scavenging activity and the reducing power of methoxy gossypols, aforementioned data sufficiently demonstrated that gossypol and its two methoxy derivatives could be used as alternative antioxidants instead of BHT within the safety limit of gossypol that is set by the regulators. In the United States, any cottonseed protein products intended for human use must contain no more than 450 ppm free gossypol as set by FDA in 1974. The Protein Advisory Group of the United Nations Food and Agriculture and World Health organizations (FAO/WHO) has set limits of 600 ppm of free gossypol and 12,000 ppm total gossypol for human consumption.

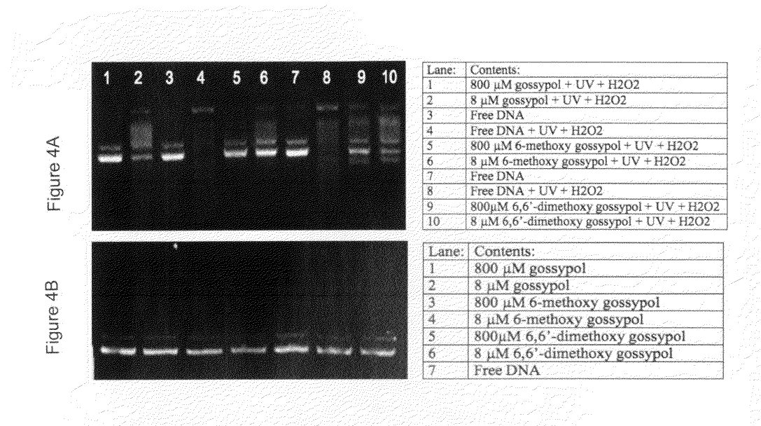

Assay of DNA Damage. Hydroxyl radicals can attack DNA to cause strand scission. Supercoiled plasmid DNA could be greatly damaged under H₂O₂ and UV induced oxidative stress, eliminating the major supercoiled band and resulting in a smear composed of nicked circles and linearized plasmids (FIGS. 4A and 4B). The plasmid DNA that was exposed to oxidative conditions in the presence of gossypol or methoxy gossypol showed less damage than the blank controls. Gossypol offered the greatest protection, followed by 6-methoxy and 6,6′-dimethoxy gossypol (FIG. 4A). This is consistent with the observation of their antioxidant capabilities in the DPPH scavenging test and reducing power test. Dose-dependent protection against oxidative DNA damage was observed for gossypol and methoxy gossypol. The higher the concentration of gossypol or methoxy derivatives, the better the DNA protection. These results indicated that gossypol and methoxy derivatives may be good DNA protectors. DNA damage by the presence of the chemical alone was also assayed (FIG. 4B). There is no apparent difference between DNA treated with chemical and untreated DNA indicating that gossypol and methoxy gossypol did not cause the DNA damage observed under oxidative stress.

Antioxidants are important to the biological systems. The normal process of oxidation could produce highly reactive free radicals, which can readily react with and damage other molecules, such as DNA. The DNA damage is correlated to some chronic disease, such as, cancer. The presence of strong antioxidants, such as gossypol or methoxy gossypol in the system, can “mop up” free radicals before the damage to other essential molecules. So gossypol and methoxy gossypol may be alternative antioxidant food additives.

Anticancer Activities. Apoptosis, a major process for cell death, plays an essential role as a protective mechanism against cancer cells. Induction of apoptosis is a highly desirable mode as a therapeutic strategy for cancer treatment. Various kinds of molecular targets have been investigated for gossypol-induced antiproliferative activity. Treatment of cancer cells with gossypol resulted in cell cycle arrest on G0/G1 phase by activation of transforming growth factor-b (15, 16), upregulation of P53 and P21, and downregulation of cyclin D1 and Rb. However no information has been provided about the anti-cancer activities of the methoxy gossypol derivatives.

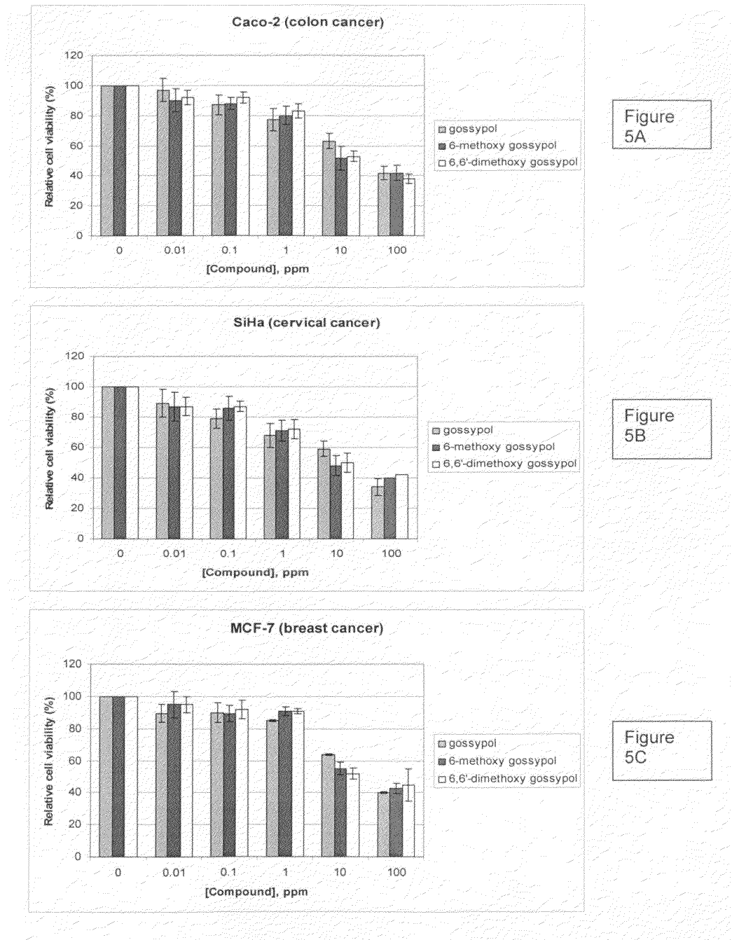

Our results showed that 6-methoxy gossypol, and 6,6′-dimethoxy gossypol had similar dose-dependent inhibitive capacity as gossypol against cervical cancer cell line, breast cancer cells line, and colon cancer cell line (FIGS. 5A, 5B, and 5C). For each cancer cell line, gossypol and methoxy gossypol, under same concentration, did not show significant difference (P>0.05) except at concentration of 10 ppm. At this concentration, 6-methoxy gossypol and 6,6′-dimethoxy gossypol showed higher cancer inhibitive activity than gossypol for all three cancer cell lines. This may be because the methyl esterification of hydroxyl groups could stabilize the compounds which may weaken the influence of serum protein and other chemicals in the medium, and enhancing the anticancer activities of these compounds.

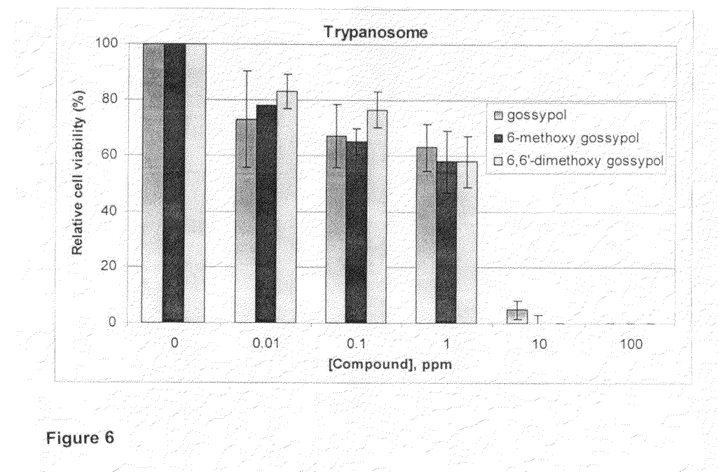

Anti-trypanosomal Activity. Trypanosomes can cause a chronic infection of sleeping sickness. It has seriously affected the health of people in the western and central African countries, and exerted significant mortality in man and livestock. Over 60 million people living in 36 sub-Saharan countries are threatened by the sleeping sickness (17) and 48000 deaths were reported in 2002 (18). In addition, 46 million cattle are exposed to the risk of the sleeping disease. The disease costs an estimated 1340 million USD per year (19). However, only a few drugs are available for the treatment of trypanosomal infections and therefore, screening of new anti-trypanosomal agents seems so important and urgent. In this study, gossypol and gossypol derivatives, methoxy gossypol were assessed for their anti-trypanosomal activities (FIG. 6). All three compounds at above 10 ppm could inhibit cell growth completely, and 1 ppm of gossypol or methoxy gossypol could inhibit 40% of trypanosome cell growth. This strong in vitro anti-trypanosomal activity of gossypol and gossypol derivatives may have potentially clinical utility for treatment of the chronic infection caused by trypanosome.

### Anti-Trypanosomal In Vivo Activity

Based upon the above data, it is Applicant's belief that the use of gossypol and its derivatives can be useful in in vivo treatment protocols for abating the symptoms and/or curing trypanosome infections. It is believed that using the following protocol with trypanosome susceptible mice will indicate that the levels of trypanosome infection of infected mice following treatment with gossypol or gossypol derivatives will be substantially reduced in comparison to untreated control mice.

Mice (BALB/c) will be infected with trypanosomes with daily monitoring to determine relative parasitemias. The susceptibility of the BALB/c mice to trypanosome infection is known as set forth in the J. Immunology 2004, May 15^{th}, 172(10):6298-303 (Ref 20) and which is incorporated herein by reference. Mice at various low, modest, and high parasitemias (approximately 1×10e5/ml, 1×10e7/ml, and 5×10e8/ml) will be treated with gossypol and gossypol derivatives at a lower 4 mg/kg body weight and a higher 20 mg/kg body weight. The gossypol will be delivered by tail vein injection and parasite levels will be monitored daily during treatment. The trypanosome strains used to inoculate the mice include the use of monomorphic trypanosome strains which are more virulent. Further, pleomorphic trypanosome strains which are less virulent will also be used to inoculate mice.

Treatement regimes may include both single treatments as well as periodic treatments. Further, the ability to supply gossypol and gossypol derivatives in a subcutaneous and gavage delivery can also be performed.

It is believed that monitoring the treated mice for trypanosomes will be indicate that gossypol and gossypol derivatives are effective in reducing the level of infection, abating symptoms caused by trypanosomes, and can lead to a complete removal of the trypanosomes from the mouse body. Further, it is expected that, following treatment, dose dependent results will be observed where higher levels of gossypol and gossypol derivatives present in the mouse's circulatory system will result in a reduction or removal of trypanosomes. It is believed that maintaining gossypol levels within the mice over a treatment regime of 3 to 10 days will result in substantial improvement of trypanosomal activity including complete removal of the trypanosomes from the infected experimentally treated mice.

In summary, gossypol and methoxy gossypol showed dose-dependent free radical scavenging activity, reducing power, oxidative DNA damage protection, anti-cancer activity, and anti-trypanosomal activity. The replacement of phenolic hydroxyl groups with methoxy groups on gossypol decreased some bioactivities in terms of the free radical scavenging activity, reducing power and the DNA damage protection ability, but methylation of the phenolic hydroxyl groups did not decrease the anti-cancer and anti-trypanosomal activities.

Set forth below under the heading, “Literature Cited” are 20 citations, the teachings and specifications of which are incorporated herein by reference for all purposes.

### LITERATURE CITED

1. Benz, C. C.; Keniry, M. A.; Ford, J. M.; Townsend, A. J.; Cox, F. W.; Palayoor, S.; Matlin, S. A.; Hait, W. N.; Cowan, K. H., Biochemical correlates of the antitumor and antimitochondrial properties of gossypol enantiomers. Mol Pharmacol 1990, 37, (6), 840-7.
2. Huang, Y. W.; Wang, L. S.; Chang, H. L.; Ye, W.; Dowd, M. K.; Wan, P. J.; Lin, Y. C., Molecular mechanisms of (−)-gossypol-induced apoptosis in human prostate cancer cells. Anticancer Res 2006, 26, (3A), 1925-33.
3. Balci, A.; Sahin, F. I.; Ekmekci, A., Gossypol induced apoptosis in the human promyelocytic leukemia cell line HL 60. Tohoku J Exp Med 1999, 189, (1), 51-7.
4. Zhang, M.; Liu, H.; Guo, R.; Ling, Y.; Wu, X.; Li, B.; Roller, P. P.; Wang, S.; Yang, D., Molecular mechanism of gossypol-induced cell growth inhibition and cell death of HT-29 human colon carcinoma cells. Biochem Pharmacol 2003, 66, (1), 93-103.
5. Dao, V. T.; Gaspard, C.; Mayer, M.; Werner, G. H.; Nguyen, S. N.; Michelot, R. J., Synthesis and cytotoxicity of gossypol related compounds. Eur J Med Chem 2000, 35, (9), 805-13.
6. Quintana, P. J.; de Peyster, A.; Klatzke, S.; Park, H. J., Gossypol-induced DNA breaks in rat lymphocytes are secondary to cytotoxicity. Toxicol Lett 2000, 117, (1-2), 85-94.
7. Percy, R. G.; Calhoun, M. C.; Kim, H. L., Seed Gossypol Variation within Gossypium barbadense L. Cotton. Crop Sci. 1996, 36, 193-197.
8. Dowd, M. K.; Pelitire, S. M., Isolation of 6-methoxy gossypol and 6,6′-dimethoxy gossypol from Gossypium barbadense Sea Island cotton. J Agric Food Chem 2006, 54, (9), 3265-70.
9. Yamaguchi, T.; Takamura, H.; Matoba, T.; Terao, J., HPLC method for evaluation of the free radical-scavenging activity of foods by using 1,1-diphenyl-2-picrylhydrazyl. Biosci Biotechnol Biochem 1998, 62, (6), 1201-4.
10. Chung, Y. C.; Chang, C. T.; Chao, W. W.; Lin, C. F.; Chou, S. T., Antioxidative activity and safety of the 50 ethanolic extract from red bean fermented by Bacillus subtilis IMR-NK1. J Agric Food Chem 2002, 50, (8), 2454-8.
11. Keum, Y. S.; Park, K. K.; Lee, J. M.; Chun, K. S.; Park, J. H.; Lee, S. K.; Kwon, H.; Surh, Y. J., Antioxidant and anti-tumor promoting activities of the methanol extract of heat-processed ginseng. Cancer Lett 2000, 150, (1), 41-8.
12. Sambrook, J.; Russell, D. W., Molecular cloning-a laboratory mannual. Cold Spring Harbor Laboratory Press: 2001.
13. Hirumi, H.; Hirumi, K., Continuous cultivation of Trypanosoma brucei blood stream forms in a medium containing a low concentration of serum protein without feeder cell layers. J Parasitol 1989, 75, (6), 985-9.
14. Nishida, J.; Kawabata, J., DPPH radical scavenging reaction of hydroxy- and methoxychalcones. Biosci Biotechnol Biochem 2006, 70, (1), 193-202.
15. Shidaifat, F.; Canatan, H.; Kulp, S. K.; Sugimoto, Y.; Zhang, Y.; Brueggemeier, R. W.; Somers, W. J.; Chang, W. Y.; Wang, H. C.; Lin, Y. C., Gossypol arrests human benign prostatic hyperplastic cell growth at G0/G1 phase of the cell cycle. Anticancer Res 1997, 17, (2A), 1003-9.
16. Shidaifat, F.; Canatan, H.; Kulp, S. K.; Sugimoto, Y.; Chang, W. Y.; Zhang, Y.; Brueggemeier, R. W.; Somers, W. J.; Lin, Y. C., Inhibition of human prostate cancer cells growth by gossypol is associated with stimulation of transforming growth factor-beta. Cancer Lett 1996, 107, (1), 37-44.
17. World Health Organization. African trypanosomiasis or sleeping sickness. 259 World Health Organ Fact Sheet. 2001,
18. World Health Organization. The world health report 2004: changing history Geneva. 2004.
19. Kristjanson P M, Swallow, B M, Rowlands G J, Kruska R L, de Leeuw P N., Measuring the costs of African animal trypanosomiasis, the potential benefits of control and returns to research. Agr Sys. 1999, (59), 79-98.
20. Sébastien Duleu, Phillippe Vincendeau, Pierrette Courtois, Silla Semballa, Isabelle Lagroye, Sylvie Daulouede, Jean-Luc Boucher, Keith T. Wilson, Bernard Veyret, and Alain P. Gobert. Mouse Strain Susceptibility to Trypanosome Infection: An Arginase-Dependent Effect. J. Immunol., May 2004; 172: 6298-6303.

What is claimed:

1. A process of treating a trypanosome infection comprising the steps of:
providing a patient infected with trypanosome;
introducing into said patient an effective amount of at least one of a gossypol, 6-methoxy gossypol, 6,6′-dimethoxy gossypol or combinations thereof, thereby removing trypanosomes from the patient.

2. A method for a treatment of a cancer selected from cervical cancer, breast cancer, and colon cancer in a mammal, which comprises administering to a mammal in need of such treatment an effective amount of a compound of formula (I) 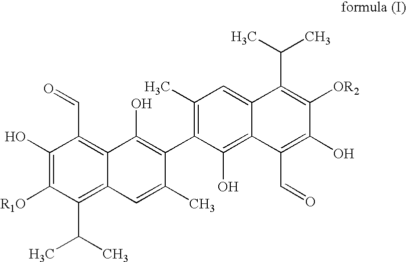
wherein R₁ and R₂ are each independently selected from the group consisting of H, and CH₃.

3. A process of treating a trypanosome infection comprising administering to a patient in need of such treatment an effective amount of a compound of formula (I) 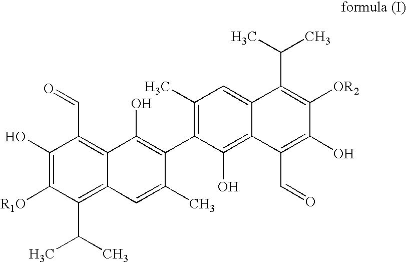
wherein R₁ and R₂ are each independently selected from the group consisting of H, and CH₃.
